(19) United States
(12) Patent Application Publication
(10) Pub. No.: US 2012/0149939 A1
(43) Pub. Date: Jun. 14, 2012

(54) Recovery of Acetic Acid from Heavy Ends in Vinyl Acetate Synthesis Process

(75) Inventors: Ilias S. Kotsianis, Houston, TX (US); Hannah E. Toomey, Houston, TX (US); Stacey Somerville, Houston, TX (US); Ismael Tejeda, Seabrook, TX (US); Alba Lala, League City, TX (US); Thomas F. Orsak, Bay City, TX (US); Brett V. Memmott, Bay City, TX (US); Kevin Hanks, League City, TX (US); Deborah R. Repman, Houston, TX (US); Victor J. Johnston, Houston, TX (US); Mark A. Deimund, Oklahoma City, OK (US)

(73) Assignee: CELANESE INTERNATIONAL CORPORATION, Dallas, TX (US)

(21) Appl. No.: 12/965,153

(22) Filed: Dec. 10, 2010

(51) Int. Cl. C07C 51/42; C07C 51/46; C07C 51/16; C07C 51/44

(52) U.S. Cl. 562/525; 562/524

(57) ABSTRACT

Recovery of acid, and in particular acetic acid, in a vinyl acetate production process. Vinyl acetate production produces heavy ends that comprise acetate containing compounds, such as monomers and polymers. Acid preferably is recovered by hydrolyzing the heavy ends and separating the resulting hydrolyzed stream to recover acid formed from the from acetate containing compounds.

### FIELD OF THE INVENTION

The present invention relates generally to processes for recovery of acetic acid from heavy ends that are formed in a vinyl acetate synthesis process and, in particular, to hydrolysis of the heavy ends to recover acetic acid.

### BACKGROUND OF THE INVENTION

Vinyl acetate is an important monomer in the production of polyvinyl acetate and polyvinyl alcohol. Vinyl acetate is conventionally produced by contacting acetic acid and ethylene with oxygen. The reaction is typically conducted in the presence of a suitable catalyst, which may comprise palladium, an alkali metal acetate promoter, and optionally a co-promoter, e.g., gold or cadmium, on a catalyst support. One exemplary vinyl acetate production process, set forth in U.S. Pat. No. 6,696,596, uses a reaction in the gas phase with oxygen or oxygen containing gasses over fixed-bed catalysts. Another example is disclosed in U.S. Pat. No. 6,040,474, which describes the manufacture of acetic acid and/or vinyl acetate using two reaction zones wherein the first reaction zone comprises ethylene and/or ethane for oxidation to acetic acid with the second reaction zone comprising acetic acid and ethylene with the product streams being subsequently separated thereby producing vinyl acetate. Also, U.S. Pat. No. 6,476,261 describes an oxidation process for the production of alkenes and carboxylic acids such as ethylene and acetic acid, which are reacted to form vinyl acetate. Each of the references mentioned above is incorporated herein by reference in its entirety.

The acetoxylation reaction, however, lends itself to the production of several unwanted by-products that include heavy ends, such as acetates, and polymers of vinyl acetate and/or ethylene. The formation of these heavy ends is detrimental in many respects. For example, the formation of these heavy ends reduces conversion of acetic acid to vinyl acetate, reduces vinyl acetate yield and may lead to vinyl acetate production equipment fouling. Thus, these heavy ends are typically removed from the vinyl acetate synthesis process via the blow down from the vaporizer. The heavy ends are typically treated as hazardous waste, thereby increasing handling costs. A heavy ends column may be used to separate acetic acid in the blow down from the heavy ends, as described in U.S. Pat. No. 6,040,474. U.S. Pat. No. 3,840,590 describes using a high-temperature wiped-film evaporator for separating residual acetic acid from the high-boiling compounds, such as acetoxyvinyl acetate and polymers. However, distilling the blow down may still produces a hazardous heavy ends waste stream.

Thus, the need exists for an efficient process for treating the heavy ends and removing acetic acid from the heavy ends, and for producing a less hazardous or non-hazardous heavy ends waste stream.

### SUMMARY OF THE INVENTION

In a first embodiment present invention is directed to a process for recovering acetic acid in a vinyl acetate synthesis process, the process comprising the steps of: providing a heavy ends stream comprising acetate compounds and derived from the vinyl acetate synthesis process; hydrolyzing at least a portion of the heavy ends stream to form a hydrolyzed stream comprising less acetate compounds than the heavy ends stream; and separating at least a portion of the hydrolyzed stream to form a vapor stream and a residue stream, wherein the vapor stream comprises acetic acid.

In a second embodiment present invention is directed to a process for recovering acetic acid in a vinyl acetate synthesis process, the process comprising the steps of: separating a heavy ends stream comprising acetate compounds from a crude vinyl acetate stream derived from the vinyl acetate synthesis process; hydrolyzing at least a portion of the heavy ends stream to form a hydrolyzed stream comprising less acetate compounds than the heavy ends stream; and separating at least a portion of the hydrolyzed stream to form a vapor stream and a residue stream, wherein the vapor stream comprises acetic acid.

In a third embodiment present invention is directed to a process for a process for recovering acetic acid in a vinyl acetate synthesis process, the process comprising the steps of providing a heavy ends stream comprising acetate compounds and derived from the vinyl acetate synthesis process; and hydrolyzing at least a portion of the heavy ends stream to form a hydrolyzed stream comprising less acetate compounds than the heavy ends stream.

### BRIEF DESCRIPTION OF DRAWINGS

The invention is described in detail below with reference to the appended drawings, wherein like numerals designate similar parts.

FIG. 1 is a schematic diagram of a vinyl acetate production process with heavy ends processing according to one embodiment of the present invention.

FIG. 2 is a schematic diagram of a heavy ends processing system with an evaporator according to one embodiment of the present invention.

FIG. 3 is a schematic diagram of a heavy ends processing system with a flasher according to one embodiment of the present invention.

FIG. 4 is a schematic diagram of a heavy ends processing system with a flasher according to one embodiment of the present invention.

FIG. 5 is a schematic diagram of a heavy ends processing system with a direct fed of blow down stream to hydrolysis reactor according to one embodiment of the present invention.

FIG. 6 is a schematic diagram of a heavy ends processing system without a heavy ends distillation column according to one embodiment of the present invention.

### DETAILED DESCRIPTION OF THE INVENTION

The present invention relates to the recovery of acetic acid from a waste stream of the vinyl acetate production process. In particular the processes may recover acetic acid from a heavy ends stream obtained from the vinyl acetate production process. The acetic acid preferably is recovered by hydrolyzing the heavy ends, in particular, acetate compounds. In addition, the present invention relates to treating the heavy ends so that the processed heavy ends may be landfillable and handled as a non-hazardous material. Recovery of acid may also advantageously increase acetic acid utilization in vinyl acetate production. This may also result in a reduction or elimination of costs associated with processing a hazardous waste stream.

In one embodiment, a stream from the vinyl acetate production process comprising heavy ends components is hydrolyzed and the resulting hydrolyzed stream is separated to recover acetic acid. The amount of acetic acid recovered by embodiments of the present invention is an improvement over conventional heavy ends processing that use a heavy ends distillation column. The recovered acetic acid may also be associated with a reduction of heavy ends that need to be treated as hazardous materials. There may be a substantially complete reduction of the hazardous heavy ends so that substantially all of the heavy ends may be treated as non-hazardous waste.

One or more evaporators may be used to separate the hydrolyzed stream to form a vapor stream that comprises the recovered acetic acid and a residue stream. Suitable evaporators may be selected from the group consisting of single stage flashers, distillation towers, short-path distillation, thin film evaporators, rising film evaporators, falling film evaporators, short tube vertical evaporators, forced circulation evaporators and combinations thereof. Preferably the evaporators are thin film evaporators, rising film evaporators, falling film evaporators, or combinations thereof, or a single stage flasher, optionally in combination with one or more of the other types of evaporators.

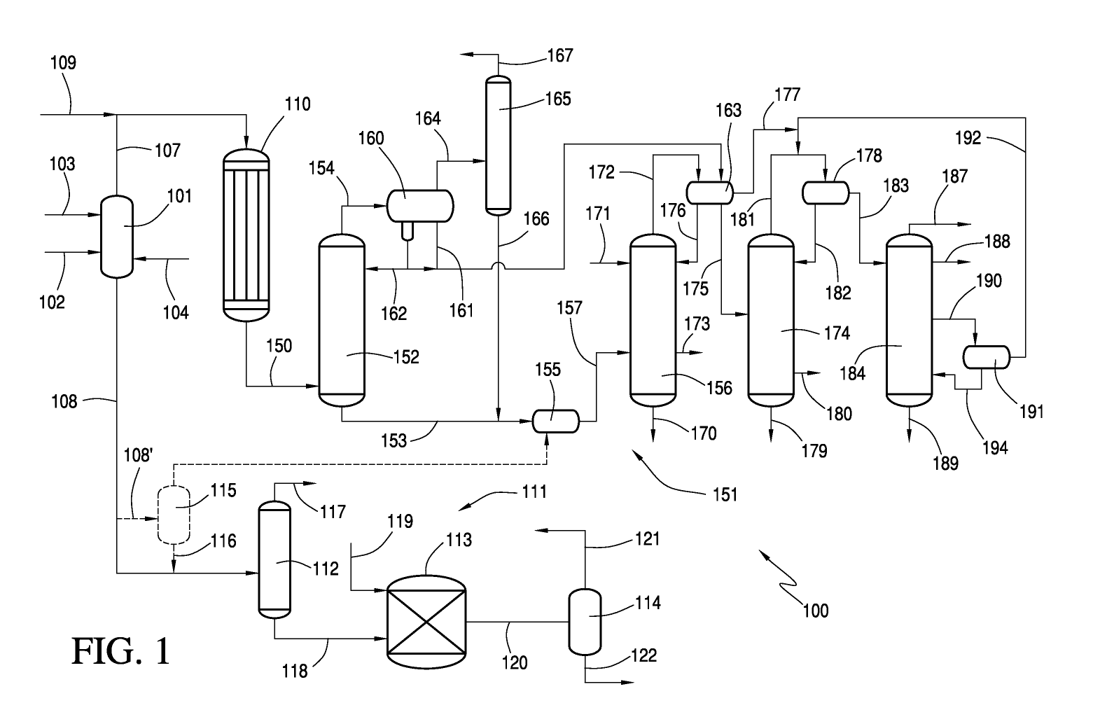

FIG. 1 illustrates an exemplary vinyl acetate synthesis system 100. Additional components and modifications may be made to the system 100 without changing the scope of the present invention. Acetic acid and ethylene are introduced to a vaporizer 101 via feed lines 102 and 103, respectively. Ethane may also be added to feed line 103. In addition, one or more recycle streams 104 may be introduced to vaporizer 101 or combined with acetic acid feed line 102. These recycle streams 104 may be withdrawn from the bottom of azeotrope column 156, dehydration column 174, and/or water stripping column 184 as well as from recovery unit 165. The temperature and pressure of vaporizer 101 may vary over a wide range. Vaporizer 101 preferably operates at a temperature from 100° C. to 200° C., e.g., from 120° C. to 150° C. The operating pressure of vaporizer 101 preferably is from 1 atm to 20 atm, e.g., 5 atm to 15 atm. Vaporizer 101 produces a vapor feed stream 107 and a blow down stream 108. Vapor feed stream 107 exits vaporizer 101 and combines with an oxygen source 109 prior to be fed to vinyl acetate reactor 110. In the production of vinyl acetate, the molar ratio of ethylene to oxygen is less than 6:1 in vinyl acetate reactor 110, e.g., less than 4:1 or 2.5:1. In the production of vinyl acetate, the molar ratio of acetic acid to oxygen is less than 6:1 in reactor 110, e.g., less than 3:1 or 2:1. In some embodiments, ethylene may be added to vapor feed stream 107 in a similar manner as the oxygen source 109. When ethylene is added to vapor feed stream 107 it is preferred that ethylene is not introduced to vaporizer 101.

Blow down stream 108 preferably is a liquid stream that comprises acetate compounds. In addition, blow down stream 108 may also comprise ethylene glycol, water, acetic acid, ethylene and/or polymers. As used herein, “acetate compounds” refers to acetate containing monomers, acetate containing oligomers and/or acetate containing polymers. Acetate containing oligomers and acetate containing polymers refer to compounds that may be hydrolyzed to produce at least one acetate containing monomer. Such oligomers and polymers include polyvinyl acetate, polyvinyl alcohol, and polymers of vinyl acetate and/or ethylene. The acetate containing monomers may comprise one or more monomers selected from the group consisting of ethylidene diacetate (ETDA), ethylene glycol monoacetate (EGMA), ethylene glycol diacetate (EGDA), vinyl acetoxy acetate (VAA), acetoxyacetic acid (AAA), cis-diacetoxyethylene (c-DAE), trans-diacetoxyethylene (t-DAE), and mixtures thereof. In one embodiment the acetate containing monomers may comprises at least AAA and one or more of the other monomers. As recycle streams 104 are fed to vaporizer 101, acetate compounds tend to build up. The build up in vaporizer 101 may require intermit cleaning which causes the process to shutdown. Without being bound by theory, it is believed that polymerization of monomers, such as ethylene and/or vinyl acetate, is induced by the presence of oxygen or oxygen-containing compounds, such as peroxides and/or oxygen radicals. The free radical oxidation of the monomers may also result in the formation of heavy ends components, e.g., polymers or acetates. In addition, the acetate compounds may react with the fresh acetic acid and ethylene that is fed to vaporizer 101.

Blow down stream 108 may also comprise impurities from acetic acid feed line 102 and/or ethylene feed line 103. In one exemplary embodiment, blow down stream 108 comprises the following primary components shown in Table 1. Other minor components, such as ethylene, ethylene glycol, glycolic acid, acetaldehyde, and water, may also be present in blow down stream 108.

TABLE 1

| | Conc. | Conc. | Conc. |
|---|---|---|---|
| Component | (Wt. %) | (Wt %.) | (Wt. %) |
| Acetic Acid | 75 to 99.5 | 85 to 99 | 90 to 96 |
| Acetate Containing Mononers | 0.1 to 25 | 0.5 to 20 | 1 to 15 |
| Acetate Containing Oligmoers | <25 | 0.5 to 20 | 1 to 15 |
| and Polymers | | | |

In one embodiment, the amount of acetate containing oligomers and polymers in the blow down stream may be less than the total amount of acetate containing monomers.

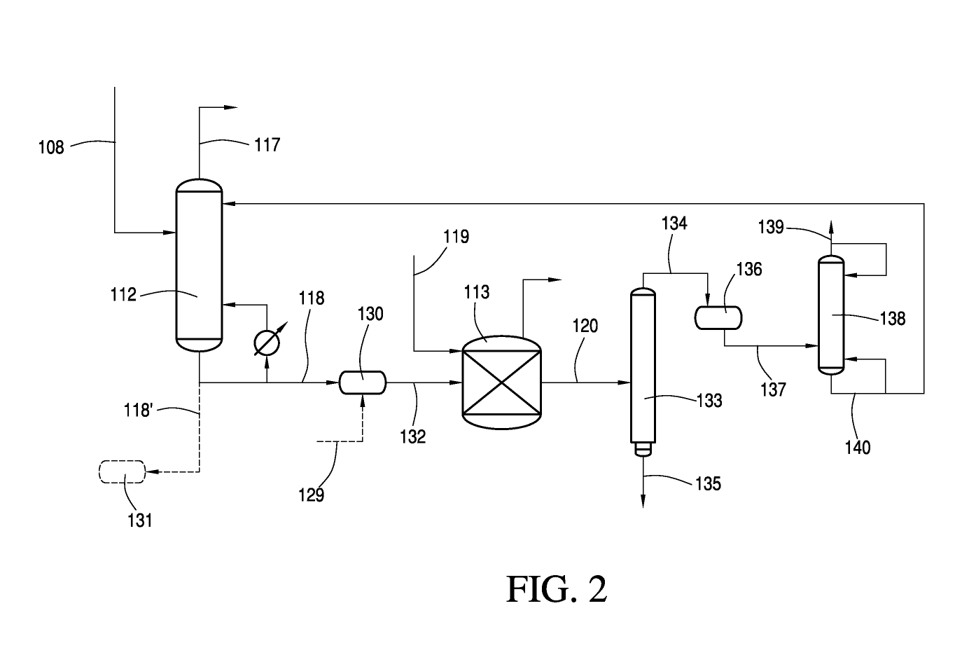

Blow down stream 108 is withdrawn from vaporizer 101 and is introduced into a heavy ends processing system 111. Exemplary heavy ends processing systems are also shown in FIGS. 2-6. In FIG. 1, the heavy ends processing system 111 comprises at least a hydrolysis reactor 113 and one or more separators 114. Heavy ends processing system 111 may also comprise a heavy ends distillation column 112 as shown in FIG. 1.

Optionally blow down stream 108 or a portion thereof 108′, may be initially separated in an optional flasher 115 to remove any light components, such as carbon dioxide, ethylene, nitrogen and other non-condensable components, in blow down stream 108. The light components may be directed upstream of azeotrope column 156, such as directed to fed line 157 of azeotrope column 156, crude tank 155, pre-dehydration column 152, recovery unit 165, reactor 110, or vaporizer 101, etc., directed to azeotrope column 156 and/or directed to residue 170 of azeotrope column 156. The liquid from flasher 115 is removed via line 116 and introduced to heavy ends distillation column 112.

Heavy ends distillation column 112 may comprise any distillation column capable of the desired separation and/or purification. Column 112 preferably comprises a tray column having from 1 to 50 trays, e.g., from 2 to 30 trays, or from 3 to 10 trays. The trays may be sieve trays, fixed valve trays, movable valve trays, or any other suitable design known in the art. In other embodiments, a packed column may be used. For packed columns, structured packing or random packing may be employed.

Heavy ends column 112 separates blow down stream 108 and optional liquid in line 116, into a distillate 117 comprising acetic acid and a residue 118 comprising a heavy ends stream. The heavy ends stream comprises acetate compounds, including monomers, oligomers, and polymers, acetic acid and mixtures thereof. Heavy ends stream may also comprise ethylene glycol, glycolic acid, and/or acetic acid. Preferably residue 118 may comprise at least 15 wt. % acetate compounds and less than 85 wt. % acetic acid.

Heavy ends column 112 may operate with no reflux, with a high reflux or near total reflux with a slip stream. Distillate 117 that is withdrawn from heavy ends column 112 may be directed any suitable location such as to azeotrope column 156, including residue 170 of column 156, or upstream of azeotrope column 156, such as directed to fed line 157 of azeotrope column 156, crude tank 155, pre-dehydration column 152, recovery unit 165, reactor 110, or vaporizer 101, etc. Optionally, distillate 117 may be further separated, preferably in a vacuum separator, before being directed to vinyl acetate synthesis system 100.

The temperature of the residue exiting in line 118 from column 112 preferably is from 100° C. to 150° C., e.g., from 105° C. to 130° C. The temperature of the distillate exiting in line 117 from column 112 preferably is from 60° C. to 105° C., e.g., from 70° C. to 95° C. The pressure of heavy ends column 112 may vary and in one embodiment, may range from 0.001 KPa to 100 KPa, e.g., from 0.001 KPa to 50 KPa or from 0.001 KPa to 35 KPa.

The heavy ends stream from residue 118 is fed to hydrolysis reactor 113, along with water stream 119, for conversion of the acetate compounds to acetic acid. Water stream 119 may be separately fed to hydrolysis reactor 113. Water stream 119 may also be combined inline with residue 118 prior to being fed to hydrolysis reactor 113. Optionally, residue 118 may be processed in one or more reboilers or evaporators before being fed to hydrolysis reactor 113. In one embodiment, the mass flow ratio of the heavy ends stream to the water stream that is fed to the hydrolysis reactor may be from 1:1 to 25:1, e.g., from 2:1 to 20:1 or from 4:1 to 10:1. In one embodiment, the water in the hydrolysis reactor is preferably less than 40 wt. % based on the total contents of the hydrolysis reactor, e.g., less than 30 wt. % or less than 20 wt. %. Preferably, the water in the hydroloysis reactor is from 10 to 25 wt. %. When water exceeds about 40 wt. % of the total contents of the hydrolysis reactor, the hydrolysis reactor may decrease in efficiency. The water in the hydrolysis reactor should be in amount that is sufficient to hydrolyze or convert at least 5% of the acetate compounds to acetic acid. Thus, the hydrolyzed stream produced by the hydrolysis reactor may contain less acetate compounds than the heavy ends stream.

In an optional embodiment, a stream comprising ethyl acetate may also be combined with residue 118 and fed to hydrolysis reactor. One such ethyl acetate stream may be the sidestream 173 withdrawn from azeotrope column 156. Sidestream 173 may also be fed to the separator 114.

Hydrolysis reactor 113 preferably is operated at a temperature of less than 200° C., e.g., less than 150° C. or less than 140° C. In terms of ranges, the temperature of hydrolysis reactor 113 may be from 100° C. to 200° C., e.g., 110° C. to 180° C., 115° C. to 165° C., or 120° C. to 150° C. Temperatures above about 180° C. may result in fouling, which reduces the amount of acetic acid that may be recovered from the heavy ends stream. The residence time in hydrolysis reactor 113 may be less than 4 hours, e.g., less than 3 hours or less than 2.5 hours. In terms of ranges, the residence time of hydrolysis reactor 113 may be from 0.5 to 4 hours, e.g., 1 to 3 hours or 2 to 3 hours. Shorter residence times of less than 0.5 are generally less preferred due to the increasing difficulty to control hydrolysis reactor 113. However, shorter residence times may be used depending on the controls for hydrolysis reactor 113. Longer residence times may also be used by increasing the size of hydrolysis reactor 113. The operating pressure of hydrolysis reactor 113 may be from 100 KPa to 1000 KPa, e.g. 130 KPa to 800 KPa or 150 KPa to 700 KPa.

In one exemplary embodiment, hydrolysis conditions favor conversion of the heavy ends streams to acetic acid at a temperature from about 125° C. to about 180° C., or preferably about 150° C., and a residence time from about 1.5 to 2.3 hours.

In a preferred embodiment of the present invention there is no catalyst present in the hydrolysis reactor 113. The hydrolysis of the acetates results in the recovery of acetic acid and corresponding derivatve compounds, such as alcohols, polyols, aldehydes, etc., under the operating conditions of the hydrolysis reactor. The absence of a catalyst in the hydrolysis reactor may improve the overall efficiency of the acid recovery because no capital is required for the catalyst. Of course, it is contemplated that in other embodiments, a catalyst, e.g., an acidic catalyst, may be employed in the hydrolysis reactor. An exemplary hydrolysis catalyst may be p-toluenesulfonic acid or methanesulfonic acid.

The reactor effluent of hydrolysis reactor 113 produces a hydrolyzed stream 120 that preferrably comprises acetic acid, water, polymers, hydrolyzed compounds of the acetate compounds, and a reduced amount of acetate compounds, in particular a reduced amount of acetate containing monomers. Hydrolyzed stream 120 may also comprise glycolic acid, ethylene glycol, and acetaldehyde. In one embodiment, hydrolyzed stream 120 comprises less than 30 wt. % acetate compounds, e.g., less than 25 wt. % or less than 15 wt. %. In terms of ranges, the acetate compounds in hydrolyzed stream 120 may be from 0.5 to 30 wt. %, e.g., from 1 to 25 wt. % or from 2 to 20 wt. %. In some embodiments, hydrolyzed stream 120 may be substantially free from acetate containing monomers. Preferably, hydrolzyed stream 120 contains less acetate compounds, especially less acetate containing monomers, than the heavy ends stream. In terms of acetate containing monomers, hydrolyzed stream 120 preferably comprises less than 30 wt. % acetate containing monomers, e.g., less than 10 wt. % or less than 5 wt. %. Heavy ends stream may contain any amount of acetate compounds, but preferably comprises at least 15 wt. % acetate compounds, e.g., at least 30 wt. % or at least 40 wt. %. Heavy ends streams that contain less than 15 wt. % acetate containing monomers may also be hydrolyzed to produced a hydrolyzed stream with less acetate compounds. Preferably, efficiency may be achieved when the heavy ends stream contains at least 5 wt. % more acetate compounds that hydrolyzed stream 120, e.g., at least 10 wt. % more or at least 15 wt. % more.

Hydrolysis reactor 113 achieves efficient conversion of acetate compounds, and in particular acetate containing monomers, from the heavy ends stream and preferably hydrolyzes or converts at least 5%, e.g., at least 10% or at least 20%, of acetate compounds of the heavy ends stream. The conversion of acetates contained in the heavy ends stream to acetic acid preferably is based on the theoretical amount of acetic acid in the heavy ends stream. The theoretical amount of acetic acid is the total amount of acetic acid that may be hydrolyzed from the acetate containing mononers in the heavy ends stream, excluding acetic acid production from oligomer and polymer degradation. When oligomers and polymers are present, the acetic acid production from polymer degradation may increase the total acetic acid conversion of the heavy ends stream. In preferred embodiments, the hydrolysis reactor efficiency may achieve significant reductions in acetate containing mononers of greater than 80%, e.g., greater than 85% or greater than 90%. It should be understood that each individual acetate containing monomer may be converted at different rates.

Hydrolyzed stream 120 is fed to separator 114, e.g., flasher, evaporator or combination, to produce a vapor stream 121 comprising acetic acid and a residue stream 122. Although one separator 114 is shown in FIG. 1, it should be understood that multiple separators, either in series or in parallel may be used to process hydrolyzed stream 120. Residue stream 122 may comprise compounds corresponding to the hydrolyzed acetates, including but not limited to acids, aldehydes, polyols, and alcohols, as well as polymers and their derivatives. For example, AAA hydrolyzes to produce one mole of acetic acid and one mole of glycolic acid. There may also be VAA present in the heavy ends stream that hydrolyzes to form AAA and acetic acid, and AAA may be further hydrolyzed. Thus, there may be an initial increase in AAA in hydrolysis reactor 113. In addition, EGMA hydrolyzes to produce one mole of acetic acid and one mole of ethylene glycol. EGDA hydrolyzes to produce two moles of acetic acid and one mole of ethylene glycol. ETDA hydrolyzes to produce two moles of acetic acid and one mole of actealdehyde. Diacetoxyethylene (DAE), both cis- and trans-, hydrolyzes to produce two moles of acetic acid and one mole of aldehyde. In some embodiments, some of the acetate containing monomers may be reduced by hydrolysis while other acetate containing monomers may increase through reversible reactions. For example, EGMA may be formed under the hydrolysis reaction conditions. Overall, there is a reduction of the total amount for all of the acetate containing monomers in the heavy ends stream.

In one embodiment, a portion of residue stream 122 may be recycled to hydrolysis reactor 113 and/or heavy ends column 112. Additionally, residue stream 122 may be further treated in one or more separate hydrolysis reactors (not shown). Either of these further treatments may be done when residue stream 122 contains acetate containing monomers that were not converted during hydrolysis.

Other components in the heavy ends streams, such as oligomers and polymers, may also be hydrolyzed. For example, polyvinyl acetate and polyvinyl alcohol may be cleaved by hydrolysis to recover acetic acid and the polymer derivative. During the hydrolysis of oligomers or polymers the polymer derivative may be an acetate containing monomer, which may be further hydrolyzed.

The derivatives, such as glycolic acid, of the acetate containing mononers may further react with polymers or other compounds in the hydrolysis reactor. This may lead to increased levels of polymer in the hydrolyzed stream, but the overall amount of acetate compounds, and in particular the amount of acetate containing monomers, may still be reduced as compared to the heavy ends stream.

Residue stream 122 may be a withdrawn as a flowable liquid, powder, sludge or slurry. In one embodiment, when a thin film or rising film evaporater separates the hydrolyzed stream, the evaporator may produce a solid or sludge. When a flasher separates the hydrolyzed stream, the flasher may produce a flowable liquid. Flowable liquid may have a dynamic viscosity at 25° C. of less than 100 cps, e.g., at less than 60 cps or less than 50 cps.

In one embodiment, vapor stream 121 or a portion thereof may be fed heavy ends column 112. Vapor stream 121 may be combined with blow down stream 108 and co-fed to heavy ends column 112. Vapor stream 121 may also be fed separately to heavy ends column 112 near the feedpoint or in the upper portion of the column above the feedpoint. Preferably any acetic acid from vapor stream 121 is separated with distillate stream 117. Distillate stream 117 or a portion thereof preferably is fed to azeotrope column 156, fed to residue 170 of azeotrope column 156, or upstream of azeotrope column 156, e.g., crude tank 155, pre-dehydration column 152, recovery unit 165, reactor 110, or vaporizer 101. In one embodiment, vapor stream 121 or a portion thereof may be directly fed to azeotrope column 156, fed to residue 170 of azeotrope column 156, or upstream of azeotrope column 156. In one embodiment, distillate stream 117 to azeotrope column 156 and vapor stream 121 may be separately fed to azeotrope column 156, upstream of column 156, and/or to residue 170.

Depending on the impurities of vapor stream 121, especially aromatic compounds, the vapor stream or a portion thereof may also be fed to vaporizer 101 and/or vinyl acetate reactor 110. In one embodiment, aromatics may be produced as a byproduct during hydrolysis and/or separation, and may be present in hydrolyzed stream 120 in low traceable amounts, generally less than less than 250 ppm. When vapor stream 121 comprises aromatics, even in amounts as low as less than 0.1 ppm, vapor stream 121 preferably is fed directly or indirectly to azeotrope column 156. When vapor stream 121 comprises essentially no detectable aromatics, vapor stream 121 or a portion thereof may also be fed to heavy ends distillation column 112, azeotrope column 156, and/or upstream of azeotrope column 156, such as to vaporizer 101 and/or vinyl acetate reactor 110. Preferably, the aromatics that are produced during hydrolysis do not increase the aromatics in the final vinyl acetate monomer produced by the process by more than 0.1 ppm.

Depending on the type of separator, such as when using evaporators, residue stream 122 may comprise non-hazardous materials that may be purged and removed from the system. This reduces the costs associated with handling the wastes from the vinyl acetate process since non-hazardous materials generally are less costly to handle. Non-hazardous materials include those that are able to met acceptable toxicity limits using, for example, Toxicity Characteristic Leaching Procedure (TCLP) analysis.

Exemplary separators to produce a vapor stream include evaporators and flashers, are described below in FIGS. 2-6.

In the production of vinyl acetate, reactor 110 produces a crude vinyl acetate stream 150 that is purified in separation system 151. The synthesis of vinyl acetate is exothermic and leads to potential increases of the polymerization rates for monomers. Once polymers are formed, the polymers may be separated from crude vinyl acetate stream 150 in separation system 151 and returned to vaporizer 101 through one or more recycle streams 104.

Any of the catalyst compositions known for the production of vinyl acetate, especially Group VIII metal catalysts, may be used in embodiments of the present invention. Suitable catalysts for the production of vinyl acetate are described, for example, in U.S. Pat. Nos. 3,743,607, 3,775,342, 5,557,014, 5,990,344, 5,998,659, 6,022,823, 6,057,260, and 6,472,556, all of which are incorporated herein by reference. Preferred catalysts comprise Pd and Au, and in some embodiments potassium acetate (KOAc). The catalysts also preferably contain a refractory support, preferably a metal oxide such as silica, silica-alumina, titania or zirconia, more preferably zirconia. The vinyl acetate reaction is generally carried out at pressures from 1 to 2.5 MPa and temperatures from 100 to 250° C.

Separation system 151 in FIG. 1 is exemplary and further separation systems used in commercial production of vinyl acetate may be used with embodiments of the present invention. Suitable separation system include those described in U.S. Pat. Nos. 6,696,596, 6,476,261, and 6,040,474, the entireties of which are incorporated herein by reference.

Crude vinyl acetate stream 150, or a portion thereof, is fed to a pre-dehydration column 152. Preferably, crude vinyl acetate stream 150 may be condensed and cooled prior to being fed to column 152. In one optional embodiment, not shown, a portion of the condensed crude vinyl acetate stream 150 may be returned to vaporizer 101. The heavy ends may be removed in blow down stream 108 and unreacted acetic acid returned to reactor 110.

Pre-dehydration column 152 separates crude vinyl acetate stream 150 into a residue 153 comprising vinyl acetate, acetic acid, and water, and a distillate 154 comprising vinyl acetate, water, acetic acid, carbon monoxide, carbon dioxide and other inert gases. Residue 153 may comprise about 10 wt. % water. Residue 153 is directed to crude tank 155 and then fed to azeotrope column 156 via line 157. In some embodiments, residue 153 may bypass crude tank and fed directly to azeotrope column 156. Distillate 154 is condensed and directed to an overhead phase separation unit 160, e.g., decanter. Conditions are desirably maintained in unit 160 to separate condensed distillate 154 into an aqueous phase 161 and an organic phase 162. At least a portion of aqueous phase 161 and at least a portion of organic phase 162 are fed to an overhead receiver 163 of azeotrope column 156. A portion of the organic phase and/or aqueous phase may be refluxed to column 152. A vent stream 164 that comprises carbon monoxide, carbon dioxide, ethylene, ethane and other noncondensable gases may be withdrawn and fed to recovery unit 165, such as a scrubber. The components, such as vinyl acetate, acetic acid, and/or water, are withdrawn via line 166 and co-fed to crude tank 155. Optionally, a portion of the residue in line 166 may be fed to pre-dehydration column 152. The vapors from recovery unit 165 comprise carbon monoxide, carbon dioxide, ethylene, ethane and mixtures thereof, and may be purged in line 167 or retained within system 100. For example, vapors in line 167, or a portion thereof, may be returned to vaporizer 101.

From crude tank 155, the crude vinyl acetate is directed via line 157 to azeotrope column 156 that separates a vinyl acetate-water azeotrope from acetic acid, as well as from ethyl acetate. Acetic acid is withdrawn as the residue in line 170. Water, or a suitable azeotropic agent, is fed to column 156 via line 171 to form an azeotrope between vinyl acetate and water that is removed as distillate 172. Ethyl acetate may also be withdrawn as a sidestream 173. Distillate 172 is fed to overhead receiver 163 along with the aqueous phase 161 and organic phase 162 from pre-dehydration column 152. In overhead receiver 163, the components may phase separate into a light phase and a heavy phase. The heavy phase comprises vinyl acetate and water and is fed to a dehydration column 174 via line 175. A portion of the light phase may be refluxed via line 176 to column 156. In addition, a portion of the light phase may be fed via line 177 to overhead receiver 178 of dehydration column 174.

The residue of azeotrope column 156 is withdrawn via stream 170 and may be removed or preferably returned to vaporizer 101. Residue 170 may be fed to a holding tank before being returned to vaporizer. Heavy ends, such as acetates and polymers, tend to build up in the bottom of azeotrope column 156. In an exemplary embodiment, stream 170 also comprises acetic acid, water, and minor portions of vinyl acetate as well as any inhibitors. In preferred embodiments, the heavy ends are fed to vaporizer 101 and taken in blow down stream 108 to heavy ends processing system 111. Optionally, a portion of residue 170 may be taken directly to heavy ends processing system 111.

Dehydration column 174 removes additional water from the contents of line 175, thus yielding purified vinyl acetate via line 180. The residue of dehydration column 174 exits via line 179 and may be recycled in part, or otherwise disposed. In other embodiments, purified vinyl acetate may be removed as the residue of the dehydration column 174. The water-containing distillate of dehydration column 174 may be directed to overhead receiver 178 via line 181. The contents of overhead receiver 178 may phase separate into an organic phase and an aqueous phase. The organic phase may be refluxed to column 174 via line 182. The aqueous phase may be directed via line 183 to water stripping column 184 to remove water.

Water stripping column 184 removes aldehydes via overhead stream 187. Overhead stream 187 comprises substantially acetaldehyde and preferably is separated as a separate byproduct for other chemical processes. Additional light ends may be removed via purge stream 188. Water is withdrawn from the bottom of column 184 via stream 189. A sidestream 190 may be withdrawn and phased in receiver 191. The organic phase in stream 192 may be fed to the overhead receiver 178 of dehydration column 174. The aqueous phase in stream 194 may be fed to the water stripping column 184 at a point below where sidestream 190 is withdrawn.

FIG. 2 illustrates a heavy ends processing system 111 according to one embodiment of the invention, in which the separator comprises an evaporator selected from the group consisting of thin film evaporators, rising film evaporators, falling film evaporators, short tube vertical evaporators, forced circulation evaporators and combinations thereof. Stream 108 is fed to heavy ends distillation column 112 in which stream 108 is separated into distillate 117 and residue 118 comprising a heavy ends stream. An optional pre-flasher shown in FIG. 1, may be also used. A portion of residue 118 may be directed to a reboiler. The remaining portion of residue 118 is fed to accumulator 130 that holds heavy ends prior to hydrolysis. In optional embodiments, a portion of residue 118 may be separated via line 118′ and directed to optional tank 131. In these optional embodiments, it may be preferred to direct hazardous waste to optional tank 131. In another optional embodiment, a stream that comprises heavy ends from another vinyl acetate production process or other chemical processing system may be fed to accumulator 130 via line 129. This may advantageously allow the heavy ends processing system 111 to process additional heavy ends components beyond those that are generated in the vinyl acetate production process. Heavy ends may be stored in accumulator 130 for several hours or days before hydrolysis. In addition, accumulator 130 allows heavy ends to be collected and transported, if necessary, to a site with a suitable hydrolysis reactor 113. Ultimately, a stream from the accumulator is withdrawn via line 132 and fed to hydrolysis reactor 113. In some embodiments, there may be one or more accumulators, as necessary. In addition, one or more vent streams may be withdrawn from each of the accumulators.

Hydrolysis of the heavy ends, in particular acetate containing monomers, occurs in hydrolysis reactor 113 as described above. Hydrolyzed stream 120 is withdrawn from hydrolysis reactor 113 and fed to an evaporator 133. In preferred embodiments, evaporator is selected from the group consisting of thin film evaporators, rising film evaporators, falling film evaporators, short tube vertical evaporators, forced circulation evaporators and combinations thereof. Suitable commercial evaporators include Artisan Rototherm E™ Thin Film Evaporator (Artisan Industries, Inc.). In preferred embodiments, the metallurgy may of the evaporator may be 2205 stainless steel, HASTELLOY™ BIB-2/B-3, HASTELLOY™ C/C-4/C-22/C-276, titanium other enhanced alloys, or combinations thereof.

In optional embodiments, when multiple evaporators are used, the evaporators may be in series or in parallel. For example, a pre-evaporating step using a rising film evaporator followed in series with a thin film evaporator may be used with embodiments of the present invention.

In the exemplary embodiment shown in FIG. 2, evaporator 133 is a thin film evaporator that produces an overhead stream 134 and a residue stream 135. In addition, one or more vent streams (not shown) may be withdrawn from the evaporator. Volatile components may be removed from hydrolyzed stream 120 in evaporator 133. The structure of the thin film evaporator may vary depending on the manufacturer without varying the scope of the present invention. A thin-film evaporator may be suitable for separating hydrolyzed stream 120 because the solid residue that accumulates may have poor flow properties and/or may be prone to agglomerating. Overhead stream 134 comprises the recovered acetic acid and other light components such as acetaldehyde. Preferably overhead stream 134 comprises minor components of acetate containing compounds and contains less acetate containing compounds than the heavy ends stream 118. Residue stream 135 comprises compounds that correspond to the hydrolyzed acetates, including but not limited to acids, aldehydes, polyols, and alcohols, as well as remaining acetate containing compounds. Residue stream 135 may be landfillable and not be non-hazardous. Evaporator 133 preferably operates at a relatively higher temperature than heavy ends distillation column 112. In one embodiment, overhead stream 134 has a temperature from 50° C. to 400° C., e.g., from 50° C. to 300° C. Evaporator 133 may operate under a wide pressure range depending on the type of evaporator and may operate at a pressure from 0.01 atm to 5 atm.

Overhead stream 134 may be furthered processed to separate light components. As shown in FIG. 2, overhead stream 134 may be condensed and introduced to an accumulator 136. Optionally, a vent stream (not shown) may be removed from accumulator 136. A stream 137 may be pumped from accumulator 136 to column 138. Column 138 removes light compounds overhead in stream 139 and acetic acid via residue 140. A portion of residue 140 may be reboiled at the bottom of column 138. Residue 140 preferably is directed to distillation column 112. In one embodiment, residue 140 may be co-fed with blow down stream 108 or separately fed at a point above feedpoint of blow down stream 108 to column 112. Residue 140 optionally may be fed directly to distillate 117. The light compounds in stream 139 may be condensed and refluxed to column 138. Optionally, a vapor portion of stream 139 may be taken and purged through a scrubber or adsorber (not shown). Optionally, residue 140 may also be fed to azeotrope column 156, residue 170 of column 156, or upstream of column 156. Additional components, such as pumps, compressors, heaters, reboilers, and chillers may be included in the system, as would be appreciated to those skilled in the art.

Although one evaporator is shown in FIG. 2, in another embodiment, hydrolyzed stream 120 may be processed in a rising film evaporator followed by a thin film evaporator. Depending on the composition of hydrolyzed stream 120, the dual evaporators may be suitable for removing residue, including solid residue, from the recovered acid.

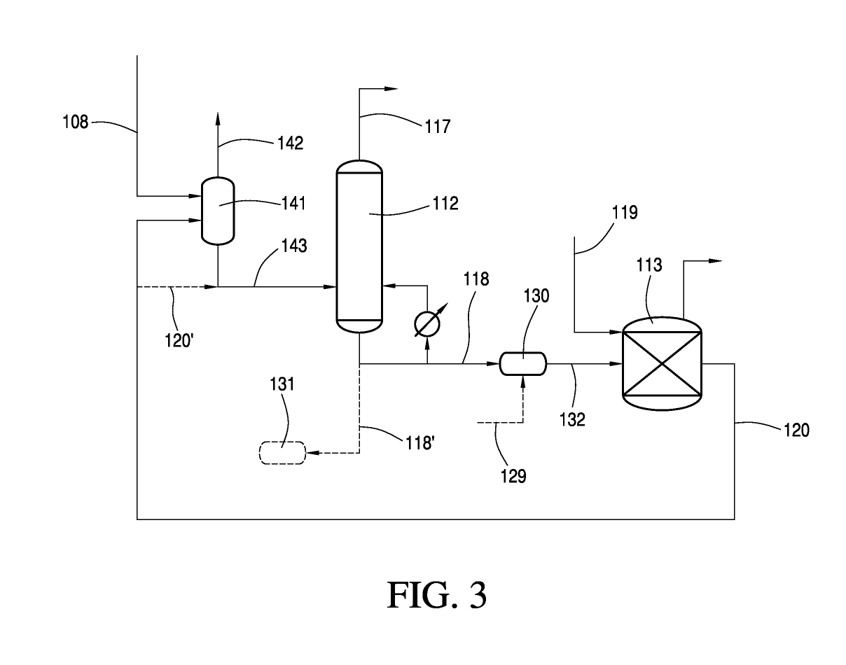

FIG. 3 illustrates one embodiment in which the separator comprises a single stage flasher. In this embodiment, the separator 114 of FIG. 1 and optional flasher 115 are combined into a single stage flasher 141. In alternative embodiments, there may be separate flashers for separating blow down stream 108 and hydrolyzed stream 120. Although one flasher is shown in FIG. 3, it should be understood that multiple flashers in series may be used in embodiments of the present invention. Blow down stream 108 may be fed to flasher 141 along with hydrolyzed stream 120 from hydrolysis reactor 113. Flasher 141 produces a vapor stream 142 that may be purged and a liquid stream 143 that is fed to heavy ends column 112. Vapor stream 142 may be purged or returned to azeotrope column 156, residue 170 of column 156, or upstream of column 156. Optionally, a portion of hydrolyzed stream may be fed to column 112 via line 120′ and bypasses flasher 141. In optional embodiments, a portion of blow down stream 108 may be fed to flasher 141 and heavy ends column 112. In some applications, a flasher may efficiently recover acetic acid from the hydrolyzed stream 120 with reduced capital intensity and may eliminate or reduce the need for solids handling.

Flashers for separating for the hydrolyzed stream preferably operate at a temperature from 50° C. to 250° C., e.g., 100° C. to 200° C. or 145° C. to 160° C. Flasher may operate at a temperature similar to the hydrolysis reactor. In addition, flashers preferably operate at a pressure from 40 KPa to 300 KPa, e.g., 105 KPa to 250 KPa or 110 KPa to 140 KPa.

In a further embodiment, a portion of residue 140 of column 138 shown in FIG. 2, may be fed to flasher 141 shown in FIG. 3.

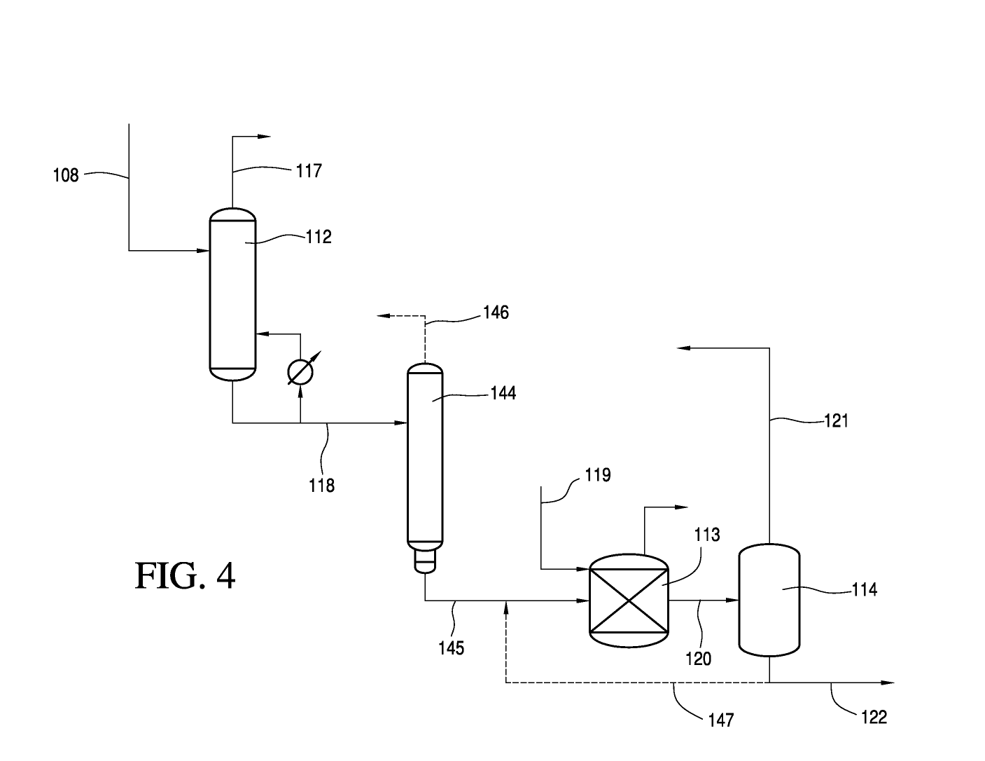

FIG. 4 illustrates another embodiment in which the separator comprises a single stage flasher. In this embodiment, blow down stream 108 is fed to heavy ends column 112 to produce a distillate stream 117 and a residue stream 118. Residue stream 118 is fed to one or more evaporators 144. For purposes of clarity one evaporator is shown in FIG. 4, however, there may be several evaporators selected from the group consisting of thin film evaporators, rising film evaporators, falling film evaporators, short tube vertical evaporators, forced circulation evaporators and combinations thereof. In one embodiment, there may be a thin film evaporator and a rising film evaporator.

Evaporator 144 produces a residue stream 145 that comprises a heavy ends stream. Residue stream 145, along with a water stream 119, is fed to hydrolysis reactor 113 under the conditions described above to produce a hydrolyzed stream 120. Hydrolyzed stream 120 is fed to a separator 114 to yield a vapor stream 121 and a liquid residue stream 122. In one embodiment, separator 114 may be one or more flashers and/or evaporators. Vapor stream 121 comprises acetic acid recovered from hydrolyzed stream 120. Separator 114 may operate at a temperature similar to the hydrolysis reactor and at a preferred pressure from 110 KPa to 140 KPa. A portion of liquid residue stream 122 may be optionally recycled via line 147 to hydrolysis reactor 113. When a portion of liquid residue stream 122 is recycled the weight ratio of the residue that is purged via stream 122 and recycled via line 153 may be from 100:1 to 1:1, e.g., from 30:1 to 1:1, or from 3:1 to 2:1.

Vapor stream 121 may be fed to heavy ends column 112 or distillate 117. In addition, vapor stream 121 may be fed to azeotrope column 156, to residue 170, or upstream of column 156. In addition, evaporator 144 also yields an optional vapor stream 146 that may be fed to heavy ends column 112 or upstream of azeotrope column 156.

FIGS. 1-4 show that blow down stream 108 from vaporizer 101 is initially fed to heavy ends column 112 before hydrolysis reactor 113. In other embodiments of the present invention, blow down stream 108 may be fed directly to hydrolysis reactor 113 or initially fed to a separator other than the heavy ends column 112, such an flasher or evaporator.

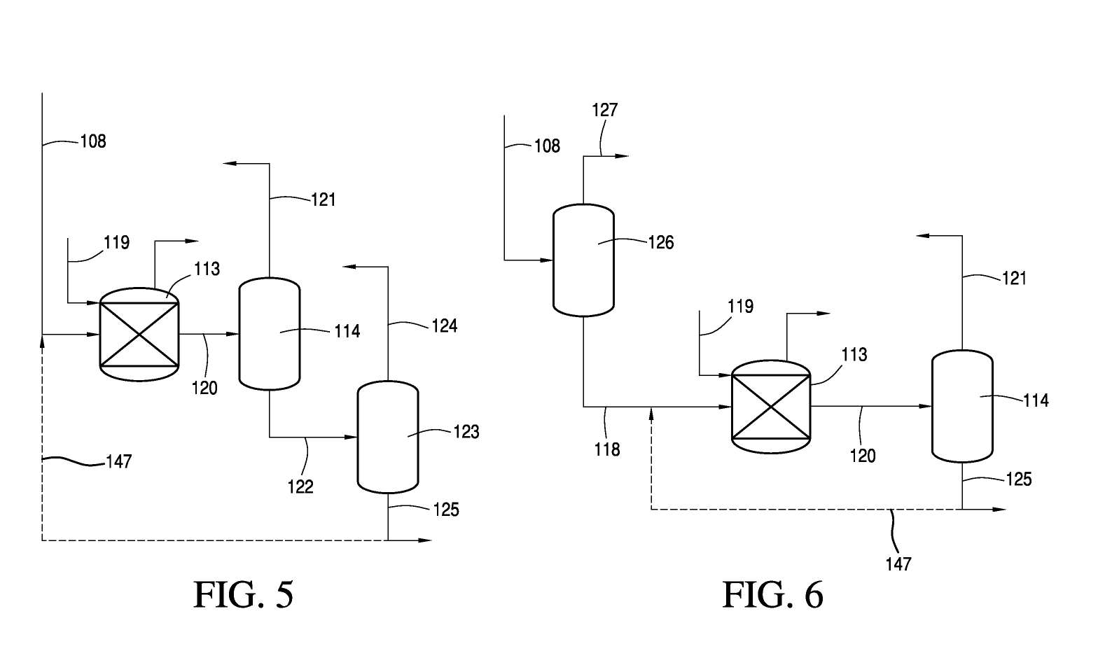

In FIG. 5 blow down stream 108 from vaporizer 101 is fed directly to hydrolysis reactor 113, along with water stream 119. In one embodiment, it may be advantageous to bypass an initial separator and hydrolyze the blow down stream directly. Blow down stream 108 may comprise a heavy ends stream that does not require an initial separation prior to the hydrolysis reaction. Optionally, a portion of blow down stream may be processed in an initial separator, such as a flasher or heavy ends column, and the remaining portion of the blow down stream fed directly to hydrolysis reactor 113. The operation conditions of hydrolysis reactor in FIG. 5 are similar to those described above. Hydrolyzed stream 120 may be processed in one or more separators 114. As shown in FIG. 5, there are two separators 114 and 123. The separators may be flashers, evaporators, or combinations thereof as described above. In one embodiment, each separator may be a vacuum flasher arranged in series. Residue stream 122 from separator 114, preferably is directed by separator 123. Vapor streams 121 and 124 comprise acetic acid recovered from heavy ends stream. Vapor streams 121 and 124 may be returned to azeotrope column 156, residue 170 of column 156, or upstream of column 156. Residue stream 125 may be purged from system. A portion of stream 125 may be optionally recycled via line 147 to hydrolysis reactor 113.

In some embodiments, blow down stream 108 may be directed to separator that is not a distillation column prior to hydrolysis reactor 113. In FIG. 6 blow down stream 108 may be initially processed in separator 126. As shown in FIG. 6, blow down stream 108 is preferably not processed in a heavy ends distillation column prior to the hydrolysis reaction. Separator 126 is preferably a single stage flasher. In one embodiment, there may be multiple separators for initially processing blow down stream 108 prior to hydrolysis reactor 113. Separator 126 produces a vapor stream 127 that may be returned to azeotrope column 156, residue 170 of column 156, or upstream of column 156. Separator 126 also produces a residue 118 that comprises a heavy ends stream. Heavy ends stream, along with water stream 119, are fed to hydrolysis reactor 113 to produce a hydrolyzed stream 120. Hydrolyzed stream 120 may be processed in one or more separators 114. The separators may be flashers, evaporators, or combinations thereof as described above. Preferably there is a combination of multiple flashers arranged in series. Vapor stream 121 comprises acetic acid recovered from heavy ends stream. Vapor stream 121 may be returned to azeotrope column 156 or upstream of azeotrope column 156. Residue stream 125 may be purged from system. A portion of residue 125 may be optionally recycled via line 147 to hydrolysis reactor 113.

In one optional embodiment, hydrolyzed stream 120 may be separated by directly feeding heavy ends distillation column 112.

In order that the invention disclosed herein may be more efficiently understood, the following Examples are provided below.

### EXAMPLES

### Example 1

A heavy ends stream from a vinyl acetate production process was withdrawn from a heavy ends column. Heavy ends stream comprised 17.5 wt. % acetic acid, 41.9 wt. % acetate containing monomers and 37.8 wt. % acetate containing polymers. Heavy ends stream also comprises acetaldehyde and ethylene glycol. In terms of acetate containing monomers there was AAA (28.4 wt. %), ETDA (1.5 wt. %), EGMA (0.3 wt. %), VAA (1.1 wt. %), EDGA (7.9 wt. %), c-DAE (1.3 wt. %) and t-DAE (1.4 wt. %).

### Example 2

The heavy ends stream from Example 1 is fed to a hydrolysis reactor, as Run A. In addition, Run B, containing 45.2 wt. % acetate containing monomers and Run C, containing 38.8 wt. % acetate containing monomers were also fed to a hydrolysis reactor. The residence time of the hydrolysis reactor was 1.9 hours. The hydrolysis reaction was conducted in the absence of a catalyst. The heavy ends to water weight ratio for Runs A and B was 3.9:1 and for Run C is 3.8:1. The temperature of the reactor varied as indicated in Table 2. Table 2 also provides a summary of the results.

In Tables 2, 3, and 4, % accountability refers to the percentage of carbon accounted for in the hydrolyzed stream from the heavy ends stream after hydrolysis. % additional HOAc refers to the HOAc produced from the hydrolysis reactor that is in addition to any HOAc in the heavy ends stream. Reaction efficiency refers to percentage of theoretical acetic acid in heavy ends stream over the actual amount of acetic acid recovered in the hydrolyzed stream. Viscosity refers to the dynamic viscosity at 25° C. of the residue separated from the hydrolyzed stream.

TABLE 2

| | Run A | Run B | Run C |
|---|---|---|---|
| Residence Time | 1.9 hours | 1.9 hours | 1.9 hours |
| HE:H₂O Ratio | 3.8:1 | 3.9:1 | 3.8:1 |
| Temperature | 145.6° C. | 154.4° C. | 167.2° C. |
| % accountability | 96.7% | 95.7% | 96.0% |
| % additional HOAc | 115.6% | 103.0% | 102.1% |
| Reaction Efficiency | 80.9% | 71.7% | 73.2% |
| Viscosity | 42.4 cps | 43.3 cps | 89.1 cps |
| % Conversions | | | |
| ETDA | 41% | 46% | 47% |
| VAA | 97% | 98% | 98% |
| EGMA | 63% | 68% | 69% |
| c-DAE | 95% | 96% | 96% |
| t-DAE | 96% | 96% | 96% |
| AAA | 75% | 81% | 83% |

As temperature increased, the viscosity of the residue stream from the flasher also increased. Overall conversions of the acetate containing monomers were similar for each run, with the exception of AAA which showed higher conversions in Runs B and C.

### Example 3

Heavy ends streams from a vinyl acetate production process were fed to a hydrolysis reactor. Runs D and E are analyzed along with Run A from Example 1. Run D contained 39.7 wt. % acetate containing monomers and Run E contained 46.2 wt. % acetate containing monomers. The hydrolysis reaction was conducted in the absence of a catalyst. The residence times for each run was varied, as indicated in Table 3. Table 3 also provides a summary of the results.

TABLE 3

| | Run D | Run A | Run E |
|---|---|---|---|
| Residence Time | 1.5 hours | 1.9 hours | 2.3 hours |
| HE:H₂O Ratio | 3.8:1 | 3.8:1 | 4.1:1 |
| Temperature | 144.7° C. | 145.6° C. | 144.2° C. |
| % accountability | 97.0% | 96.7% | 96.7% |
| % additional HOAc | 122.6% | 115.6% | 87.4% |
| Reaction Efficiency | 77.9% | 80.9% | 61.1% |
| Viscosity | 34.0 cps | 42.4 cps | 36.7 cps |
| % Conversions | | | |
| ETDA | 38% | 41% | 47% |
| VAA | 97% | 97% | 98% |
| EGMA | 63% | 63% | 68% |
| c-DAE | 94% | 95% | 96% |
| t-DAE | 95% | 96% | 97% |
| AAA | 73% | 75% | 79% |

At lower residence times, Runs D and A more than doubled the amount of acetic acid recovered. In addition, at higher residence times there was a slight improvement in conversions from Run D to Run E.

### Example 4

Heavy ends stream from a vinyl acetate production process were fed to a hydrolysis reactor. The hydrolysis reaction was conducted in the absence of a catalyst. Runs F and G are analyzed along with Run A from Example 1. Run F contained 44.2 wt. % acetate containing monomers and Run G contained 42.6 wt. % acetate containing monomers. The water concentrations in the hydrolysis reactor of each run was varied, as indicated in Table 4. Run F had about 10 wt. % water, Run A had about 20 wt. % water, and Run G had about 30 wt. % water. Table 4 also provides a summary of the results.

TABLE 4

| | Run F | Run A | Run G |
|---|---|---|---|
| Residence Time | 1.9 hours | 1.9 hours | 1.9 hours |
| HE:H₂O Ratio | 9.6:1 | 3.8:1 | 2.6:1 |
| Temperature | 147.2° C. | 145.6° C. | 145.0° C. |
| % accountability | 97.6% | 96.7% | 98.6% |
| % additional HOAc | 62.1% | 115.6% | 132.5% |
| Reaction Efficiency | 40.2% | 80.9% | 93.6% |
| Viscosity | 48.6 cps | 42.4 cps | 23.3 cps |
| % Conversions | | | |
| ETDA | 49% | 41% | 39% |
| VAA | 95% | 97% | 99% |
| EGMA | 52% | 63% | 75% |
| c-DAE | 92% | 95% | 97% |
| t-DAE | 92% | 96% | 98% |
| AAA | 64% | 75% | 85% |

At higher water concentration in Runs A and G, the conversion of the acetate containing monomers, including AAA, improve over Run F. High conversions of acetic acid were also observed in Runs A and G. In comparison to residence time from Example 3, the increased amounts of water demonstrated a larger effect on acetate containing monomer conversions.

### Example 5

Run A was further processed in a flasher to obtain an overhead vapor stream. The hydrolyzed stream was separated in a flasher operating a temperature similar to the hydrolysis reactor and at a pressure of 5 psig (˜135 KPa). The overhead vapor stream was returned upstream of azeotrope column. The composition, in wt. %, of the overhead vapor stream is shown in Table 5 below.

TABLE 5

| | | | | | | Acetate | Acetate |
|---|---|---|---|---|---|---|---|
| | | | | | | Contain- | Contain- |
| | | | | | Glycolic | ing | ing |
| | HOAc | Water | AcH | EG | Acid | Monomers | Polymers |
| Run | 58.0 | 35.5 | 1.9 | 0.1 | 0.0 | 3.0 | 1.6 |
| A | | | | | | | |
| (wt. | | | | | | | |
| %) | | | | | | | |

Of the acetate containing monomers, the total amount of AAA and VAA is less than 0.05 wt. %.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

We claim:

1. A process for recovering acetic acid in a vinyl acetate synthesis process, the process comprising the steps of:
providing a heavy ends stream comprising acetate compounds and derived from the vinyl acetate synthesis process;
hydrolyzing at least a portion of the heavy ends stream to form a hydrolyzed stream comprising less acetate compounds than the heavy ends stream; and
separating at least a portion of the hydrolyzed stream to form a vapor stream and a residue stream, wherein the vapor stream comprises acetic acid.

2. The process of claim 1, wherein the acetate compounds comprise monomers selected from the group consisting of ethylidene diacetate, ethylene glycol monoacetate, ethylene glycol diacetate, vinyl acetoxy acetate, acetoxyacetic acid, cis-diacetoxyethylene, trans-diacetoxyethylene, and mixtures thereof.

3. The process of claim 1, wherein the acetate compounds comprise acetate containing oligomers, acetate containing polymers, and mixtures thereof.

4. The process of claim 1, wherein the separating step comprises flashing at least a portion of the hydrolyzed stream to form the vapor stream and the residue stream that is a flowable liquid.

5. The process of claim 1, wherein the separating step comprises evaporating at least a portion of the hydrolyzed stream to form the vapor stream and the residue stream that is a solid, powder, or sludge.

6. The process of claim 5, wherein the at least a portion of the hydrolyzed stream is evaporated in one or more evaporators.

7. The process of claim 6, wherein the one or more evaporators are selected from the group consisting of single stage flashers, distillation towers, short-path distillation, thin film evaporators, rising film evaporators, falling film evaporators, short tube vertical evaporators, forced circulation evaporators and combinations thereof.

8. The process of claim 1, further comprising:
vaporizing acetic acid and ethylene in a vaporizer to form a vaporized feed stream and a blow down stream; and
separating at least a portion of the blow down stream in a heavy ends column into a distillate and a residue, wherein the distillate comprises acetic acid, and wherein the residue comprises the heavy ends stream.

9. The process of claim 8, wherein the blow down stream comprises acetate compounds in an amount from 0.1 to 25 wt. %, and acetic acid in an amount from 75 to 99.5 wt. %.

10. The process of claim 8, further comprising:
reacting the acetic acid with the ethylene under conditions effective to form a crude vinyl acetate product comprising vinyl acetate;
directing the crude vinyl acetate product to a separation system for forming a vinyl acetate product stream, wherein the separation system comprises an azeotrope column for separating vinyl acetate-water azeotrope from acetic acid; and
directing at least a portion of the vapor stream to the azeotrope column, upstream of the azeotrope column, and/or residue of azeotrope column.

11. The process of claim 8, further comprising:
directing at least a portion of the distillate from the heavy ends column to the azeotrope column, upstream of the azeotrope column, and/or residue of azeotrope column.

12. The process of claim 8, further comprising directing at least a portion of the vapor stream to the heavy ends column.

13. The process of claim 8, further comprising directing at least a portion of the vapor stream to the vaporizer.

14. The process of claim 1, wherein the heavy ends stream comprises at least 15 wt. % acetate compounds and wherein the hydrolyzed stream comprises less than 30 wt. % acetate compounds.

15. The process of claim 1, wherein the heavy ends stream comprises from 15 to 80 wt. % acetate compounds and wherein the hydrolyzed stream comprises from 0.5 to 30 wt. % acetate compounds.

16. The process of claim 1, wherein the heavy ends stream comprises at least 5 wt. % more acetate compounds than the hydrolyzed stream.

17. The process of claim 1, wherein the hydrolyzing step is performed at a temperature from 100° C. to 200° C.

18. The process of claim 1, wherein the hydrolyzing step is performed with a residence time of less than 4 hours.

19. The process of claim 1, wherein the hydrolyzing step is performed in a hydrolysis reactor.

20. The process of claim 19, further comprising feeding water to the hydrolysis reactor, wherein the mass flow ratio of the at least a portion of the heavy ends stream to the water that is fed to the hydrolysis reactor is from 1:1 to 25:1.

21. The process of claim 19, wherein the hydrolysis reactor contains water in an amount less than 40 wt. %.

22. The process of claim 19, wherein the hydrolysis reactor contains no catalyst.

23. A process for recovering acetic acid in a vinyl acetate synthesis process, the process comprising the steps of:
separating a heavy ends stream comprising acetate compounds from a blow down stream derived from the vinyl acetate synthesis process;
hydrolyzing at least a portion of the heavy ends stream to form a hydrolyzed stream comprising less acetate compounds than the heavy ends stream; and
separating at least a portion of the hydrolyzed stream to form a vapor stream and a residue stream, wherein the vapor stream comprises acetic acid.

24. The process of claim 23, wherein the heavy ends stream comprises at least 15 wt. % acetate compounds and wherein the hydrolyzed stream comprises less than 30 wt. % acetate compounds.

25. The process of claim 22, wherein the heavy ends stream comprises from 15 to 80 wt. % acetate compounds and wherein the hydrolyzed stream comprises from 0.5 to 30 wt. % acetate compounds.

26. The process of claim 23, wherein the separating of the at least a portion of the hydrolyzed stream comprises flashing at least a portion of the hydrolyzed stream to form the vapor stream and the residue stream that is a flowable liquid.

27. The process of claim 23, wherein the separating step comprises evaporating at least a portion of the hydrolyzed stream to form the vapor stream and the residue stream that is a solid, powder, or sludge.

28. The process of claim 23, wherein the at least a portion of the hydrolyzed stream is evaporated in one or more evaporators.

29. The process of claim 28, wherein the one or more evaporators are selected from the group consisting of single stage flashers, distillation towers, thin film evaporators, rising film evaporators, falling film evaporators, short tube vertical evaporators, forced circulation evaporators and combinations thereof.

30. The process of claim 23, further comprising:
vaporizing acetic acid and ethylene in a vaporizer to form a vaporized feed stream and the blow down stream; and
separating at least a portion of the blow down stream in a heavy ends column into a distillate and a residue, wherein the distillate comprises acetic acid, and wherein the residue comprises the heavy ends stream.

31. The process of claim 30, further comprising:
reacting the acetic acid with the ethylene under conditions effective to form a crude vinyl acetate product comprising vinyl acetate;
directing the crude vinyl acetate product to a separation system for forming a vinyl acetate product stream, wherein the separation system comprises an azeotrope column for separating vinyl acetate-water azeotrope from acetic acid; and
directing at least a portion of the vapor stream to the azeotrope column, upstream of the azeotrope column, and/or residue of azeotrope column.

32. The process of claim 31, further comprising:
directing at least a portion of the distillate from the heavy ends column to the azeotrope column, upstream of the azeotrope column, and/or residue of azeotrope column.

33. The process of claim 30, further comprising directing at least a portion of the vapor stream to the heavy ends column.

34. The process of claim 30, further comprising directing at least a portion of the vapor stream to the vaporizer.

35. The process of claim 23, wherein the heavy ends stream comprises at least 15 wt. % acetate compounds and wherein the hydrolyzed stream comprises less than 30 wt. % acetate compounds.

36. The process of claim 23, wherein the heavy ends stream comprises from 15 to 80 wt. % acetate compounds and wherein the hydrolyzed stream comprises from 0.5 to 30 wt. % acetate compounds.

37. The process of claim 23, wherein the heavy ends stream comprises at least 5 wt. % more acetate compounds than the hydrolyzed stream.

38. The process of claim 23, wherein the heavy ends stream comprises acetate compounds in an amount from 15 to 80 wt. %, and acetic acid in an amount from 1 to 50 wt. %.

39. The process of claim 23, wherein the acetate compounds comprise monomers selected from the group consisting of ethylidene diacetate, ethylene glycol monoacetate, ethylene glycol diacetate, vinyl acetoxy acetate, acetoxyacetic acid, cis-diacetoxyethylene, trans-diacetoxyethylene, and mixtures thereof.

40. The process of claim 23, wherein the acetate compounds comprise acetate containing oligomers, acetate containing polymers, and mixtures thereof.

41. The process of claim 23, wherein the hydrolyzing step is performed at a temperature from 100° C. to 200° C.

42. The process of claim 23, wherein the hydrolyzing step is performed with a residence time of less than 4 hours.

43. The process of claim 23, wherein the hydrolyzing step is performed in a hydrolysis reactor.

44. The process of claim 43, further comprising feeding water to the hydrolysis reactor, wherein the mass flow ratio of the at least a portion of the heavy ends stream to the water that is fed to the hydrolysis reactor is from 1:1 to 25:1.

45. The process of claim 43, wherein the hydrolysis reactor contains water in an amount less than 40 wt. %.

46. The process of claim 43, wherein the hydrolysis reactor contains no catalyst.

47. A process for recovering acetic acid in a vinyl acetate synthesis process, the process comprising the steps of:
providing a heavy ends stream comprising acetate compounds and derived from the vinyl acetate synthesis process; and
hydrolyzing at least a portion of the heavy ends stream to form a hydrolyzed stream comprising less acetate compounds than the heavy ends stream.
